(19) United States
(12) Patent Application Publication
(10) Pub. No.: US 2008/0095732 A1
(43) Pub. Date: Apr. 24, 2008

(54) Personal care compositions

(76) Inventor: Rosemarie Osborne, Oxford, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/893,468

(22) Filed: Aug. 16, 2007

Related U.S. Application Data
(63) Continuation-in-part of application No. 11/411,285, filed on Apr. 26, 2006.
(60) Provisional application No. 60/675,274, filed on Apr. 27, 2005.

(51) Int. Cl. A61K 8/64; A61K 38/08; A61Q 19/00; A61Q 5/00

(52) U.S. Cl. 424/70.14; 514/17

(57) ABSTRACT

Personal care compositions comprising a dipeptide; one or more of the following: a pentapeptide, a second dipeptide, a vitamin B3 compound, a hexamindine compound, and/or a sugar amine; and a dermatologically acceptable carrier. Methods of using such compositions to treat the condition of keratinous tissue. In certain embodiments, the C terminal amino acid of said dipeptide is threonine.

### CROSS-REFERENCE TO RELATED APPLICATION

This application is a continuation-in-part of U.S. application Ser. No. 11/411,285 filed Apr. 26, 2006, which claims the benefit of priority to U.S. Provisional Application No. 60/675,274, filed Apr. 27, 2005. Both U.S. application Ser. No. 11/411,285 and U.S. Provisional Application No. 60/675,274 are herein incorporated by reference.

### FIELD OF THE INVENTION

The present invention relates to personal care compositions comprising a dipeptide and other ingredients. Such compositions are useful for regulating the condition of mammalian keratinous tissue (e.g., skin, hair, and/or nails), especially regulating the cosmetic appearance of mammalian keratinous tissue.

### BACKGROUND OF THE INVENTION

Many personal care products currently available to consumers are directed primarily to improving the health and/or physical appearance of the skin, hair, or nails. Among these skin, hair, or nail care products, many are directed to delaying, minimizing or even eliminating skin, hair, or nail changes typically associated with the aging or the environmental damage to human skin, hair, or nails. Numerous compounds have been described in the art as being useful for regulating skin, hair, or nail condition.

Skin, hair, and nails are subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin, hair, or nails. Whether extrinsic or intrinsic, these factors result in visible signs of skin, hair, and nail aging and environmental damage (e.g., such as sunlight damage, smoke damage, and damage from pollutants such as nitrogen oxides, sulfur oxides, ozone, and metals such as lead). To many people, the loss of the attractiveness of skin, hair, or nails is a reminder of the disappearance of youth. As a result, the maintenance of a youthful appearance has become a booming business in youth-conscious societies. Numerous products and treatments are available in various forms to help maintain the appearance of younger hair, skin, and nails.

Extrinsic or intrinsic factors may result in the thinning and general degradation of the skin, hair, or nails. For example, as the skin, hair, and nails naturally age, there is a reduction in the cells and blood vessels that supply the skin, hair, or nails. There is also a flattening of the dermal-epidermal junction which results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990.

A large number of skin, hair, and nail care actives are known in the art and used to improve the health and/or cosmetic appearance of the skin, hair, or nails. For instance, various peptides are included in skin, hair, and nail care compositions to provide skin, hair, or nail care benefits. However, not all peptides can provide the benefits desired.

For instance, C terminal serine residues can yield dipeptides which may not be dermopharmaceutically and/or cosmetically active or which may not be useful in preferred applications. For instance, dipeptides including, for example, lysine and serine (Lys-Ser) can have inadequate properties for many dermopharmaceutical and cosmetic applications. Thus, it would be desirable to provide personal care compositions comprising a dipeptide that can provide superior properties when compared to the corresponding Lys-Ser dipeptide.

### SUMMARY OF THE INVENTION

The present invention provides personal care compositions comprising a dipeptide that can provide superior properties when compared to the corresponding Lys-Ser dipeptide. The dipeptide of the present invention is a dipeptide wherein the C terminal amino acid is threonine (“Thr”). The N terminal amino acid of such dipeptide may be a basic amino acid, These include the naturally occurring amino acids lysine (Lys), arginine (Arg) and histidine (His). Thus, a suitable dipeptide in accordance with the present invention has the sequence Lys-Thr and N-acyl derivatives and esters, and nitrogen containing C terminal derivatives thereof.

The personal care compositions comprise one or more of such dipeptides and/or derivatives of such dipeptides, preferably in a safe and effective amount. The personal care compositions comprise one or more of the following: a pentapeptide, a second peptide other than said dipeptide wherein the C terminal amino acid is threonine (“Thr”), a vitamin B3 compound, a hexamindine compound, and/or a sugar amine.

The present invention also relates to methods of using such compositions to regulate the condition of mammalian keratinous tissue (e.g., skin, hair, or nails). Said methods generally comprise the step of topically applying a composition of the present invention to the keratinous tissue (e.g., skin, hair, or nails) of a mammal in need of such treatment.

These and other features, aspects, and advantages of the present invention will become evident to those skilled in the art from a reading of the present disclosure.

### DETAILED DESCRIPTION OF THE INVENTION

While the specification concludes with the claims particularly pointing out and distinctly claiming the invention, it is believed that the present invention will be better understood from the following description.

As used herein, the singular term “dipeptide” is broad enough to include one or more dipeptides, dipeptide derivatives, or combinations thereof. Thus, the terms “dipeptide”, “dipeptides”, and “derivatives of dipeptides” are used interchangeably throughout. “Dipeptide” refers to both naturally occurring dipeptides and synthesized dipeptides, including naturally occurring and commercially available compositions that contain at least one dipeptide.

As used herein, the singular term “peptide” is broad enough to include one or more peptides, peptide derivatives, or combinations thereof. The term “peptide” refers to peptides containing ten or fewer amino acids and their derivatives, isomers, and complexes with other species such as metal ions (e.g., copper, zinc, manganese, magnesium, and the like). Thus, the terms “peptide”, “peptides”, and “derivatives of peptides” are used interchangeably throughout. “Peptide” refers to both naturally occurring peptides and synthesized peptides, including naturally occurring and commercially available compositions that contain at least one peptide.

All percentages and ratios used herein are by weight of the total composition and all measurements made are at 25° C., unless otherwise designated.

The term “keratinous tissue,” as used herein, refers to keratin-containing layers disposed as the outermost protective covering of mammals (e.g., humans, dogs, cats, etc.) which includes, but is not limited to, skin, mucosa, lips, hair, toenails, fingernails, cuticles, hooves, etc.

The terms “topical application”, “topically”, and “topical”, as used herein, mean to apply (e.g., spread, spray) the compositions of the present invention onto the surface of the keratinous tissue.

The term “dermatologically acceptable,” as used herein, means that the compositions or components thereof so described are suitable for use in contact with mammalian keratinous tissue without undue toxicity, incompatibility, instability, allergic response, and the like.

As used herein, “effective amount” means an amount of a compound or composition sufficient to significantly induce a positive keratinous tissue benefit, including independently or in combination with other benefits disclosed herein. This means that the content and/or concentration of dipeptide in the formulation is sufficient that when the formulation is applied with normal frequency and in a normal amount, the formulation can result in the treatment of one or more undesired keratinous tissue conditions (e.g., skin wrinkles). For instance, the amount can be an amount sufficient to inhibit or enhance some biochemical function occurring within the keratinous tissue. This amount of dipeptide may vary depending upon the type of product, the type of keratinous tissue condition to be addressed, and the like.

The term “safe and effective amount” as used herein means an amount of a compound or composition sufficient to significantly induce a positive benefit, preferably a positive keratinous tissue appearance or feel benefit, including independently or in combinations with the benefits disclosed herein, but low enough to avoid serious side effects, i.e., to provide a reasonable benefit to risk ratio, within the scope of sound judgment of the skilled artisan.

The personal care compositions of the present invention can be useful for treating keratinous tissue (e.g., hair, skin, or nails) condition. As use herein, “treating” or “treatment” or “treat” includes regulating and/or immediately improving keratinous tissue cosmetic appearance and/or feel. As used herein, “regulating” or “regulation” means maintaining or improving the health and/or cosmetic appearance, and includes both prophylactically regulating and/or therapeutically regulating. Regulation of keratinous tissue condition, namely mammalian and in particular human skin, hair, or nail condition, is often required due to conditions which may be induced or caused by factors internal and/or external to the body. Examples include environmental damage, radiation exposure (including ultraviolet radiation), chronological aging, menopausal status (e.g., post-menopausal changes in skin, hair, or nails), stress, diseases, disorders, etc. For instance, “regulating skin, hair, or nail condition” includes prophylactically regulating and/or therapeutically regulating skin, hair, or nail condition, and may involve one or more of the following benefits: thickening of skin, hair, or nails (e.g, building the epidermis and/or dermis and/or sub-dermal [e.g., subcutaneous fat or muscle] layers of the skin, and where applicable the keratinous layers of the nail and hair shaft) to reduce skin, hair, or nail atrophy, increasing the convolution of the dermal-epidermal border (also known as the rete ridges), preventing loss of skin or hair elasticity (loss, damage and/or inactivation of functional skin elastin) such as elastosis, sagging, loss of skin or hair recoil from deformation; melanin or non-melanin change in coloration to the skin, hair, or nails such as under eye circles, blotching (e.g., uneven red coloration due to, e.g., rosacea) (hereinafter referred to as “red blotchiness”), sallowness (pale color), discoloration caused by telangiectasia or spider vessels, and graying hair.

As used herein, prophylactically regulating keratinous tissue condition includes delaying, minimizing and/or preventing visible and/or tactile discontinuities in keratinous tissue (e.g., texture irregularities in the skin, hair, or nails which may be detected visually or by feel), including signs of skin, hair, or nail aging. This is also encompassed within the term “treating.”

As used herein, therapeutically regulating keratinous tissue condition includes ameliorating, e.g., diminishing, minimizing and/or effacing, discontinuities in keratinous tissue (e.g., skin, hair, or nails). This is also encompassed within the term “treating.”

As used herein, “personal care composition” means a composition in a form intended for topical application to keratinous tissue for the purpose of treating keratinous tissue (e.g., skin, hair, nails), and not intended for subsequent manufacture or modification.

The compositions of the present invention can also be useful for immediately improving keratinous tissue (e.g., skin, hair, or nail) cosmetic appearance and/or feel. For example, topical compositions of the present invention can be useful for regulating the cosmetic appearance of skin, hair, or nail condition by providing an immediate visual improvement in skin, hair, or nail appearance following application of the composition to the skin, hair, or nails. Generally speaking, topical compositions of the present invention which further contain particulate materials (e.g., pigments) can be most useful for providing immediate visual improvement.

The term “sagging” as used herein means the laxity, slackness, or the like condition of skin that occurs as a result of loss of, damage to, alterations to, and/or abnormalities in dermal elastin, muscle and/or subcutaneous fat.

The terms “smoothing” and “softening” as used herein mean altering the surface of the keratinous tissue such that its tactile feel is improved.

“Signs of keratinous tissue aging” include, but are not limited to, all outward visibly and tactilely perceptible manifestations as well as any other macro or micro effects due to keratinous tissue aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, fine lines, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues (e.g., fat and/or muscle), especially those proximate to the skin.

The compositions of the present invention are described in detail hereinafter.

### I. PERSONAL CARE COMPOSITIONS

In one aspect, the personal care compositions of the present invention comprise a dipeptide where the C terminal amino acid is threonine; a pentapeptide, a second peptide other than said dipeptide wherein the C terminal amino acid is threonine (“Thr”), a vitamin B3 compound, a hexamindine compound, and/or a sugar amine; and a dermatologically acceptable carrier.

The personal care compositions of the present invention can be in any suitable form. All forms of topical personal care compositions comprising these dipeptides are contemplated and can include, for instance, creams, gels, lotions, emulsions, serums, colloids, solutions, suspensions, ointments, milks, sprays, capsules, tablets, liquids, sticks, solids, pastes, powders, compacts, pencils, spray-on formulations, brush-on formulations, cloths, wipes, and the like.

Non-limiting examples of topical personal care compositions can include, without limitation, lipstick, mascara, rouge, foundation, blush, eyeliner, lipliner, lip gloss, facial or body powder, sunscreens and blocks, nail polish, mousse, sprays, styling gels, nail conditioner, bath and shower gels, shampoos, conditioners, cream rinses, hair dyes and coloring products, leave-on conditioners, sunscreens and sunblocks, lip balms, skin conditioners, cold creams, moisturizers, hair sprays, soaps, body scrubs, exfoliants, astringents, depilatories and permanent waving solutions, antidandruff formulations, antisweat and antiperspirant compositions, shaving, preshaving and after shaving products, moisturizers, deodorants, cold creams, cleansers, skin gels, and rinses. Furthermore, the composition can be applied topically through the use of a patch or other delivery device. Delivery devices can include, but are not limited to, those that can be heated or cooled, as well as those that utilize iontophoresis or ultrasound.

A. Dipeptide

The compositions of the present invention comprise a dipeptide active. As used herein, the term “dipeptide” is broad enough to include one or more dipeptides, one or more derivatives of dipeptides, and combinations thereof. Preferably, the compositions comprise an effective amount, preferably a safe and effective amount, of such dipeptide.

A suitable peptide active for use herein is the dipeptide lys-thr and derivatives thereof. A preferred dipeptide derivative-containing composition is palmitoyl-lys-thr from Sederma, France. The use of threonine (Thr) as the C terminal residue in a dipeptide is particularly desirable, and can provide superior attributes in comparison to similar dipeptides terminating with a serine. For instance, the dipeptide Lys-Thr and N-acyl derivatives and esters, and nitrogen containing C terminal derivatives thereof can provide superior properties when compared to the corresponding Lys-Ser dipeptide.

Thus, the dipeptide of the present invention is a dipeptide where the C terminal amino acid is threonine (“Thr”). More preferably, the N terminal amino acid of such dipeptides is a basic amino acid, one which is positively charged at a pH of 6.0. These include the naturally occurring amino acids lysine (Lys), arginine (Arg) and histidine (His). Most preferred is the use of lysine. Thus, a particularly preferred dipeptide in accordance with the present invention has the sequence Lys-Thr and N-acyl derivatives and esters, and nitrogen containing C terminal derivatives thereof.

Dipeptides and derivatives in accordance with the present invention include, without limitation, His-Thr, Arg-Thr, Lys-Thr, Alk-His-Thr, Alk-Arg-Thr, Alk-Lys-Thr, His-Thr-OAlk, Arg-Thr-OAlk, Lys-Thr-OAlk, His-Thr-NR₁R₂, Arg-Thr-NR₁R₂, Lys-Thr-NR₁R₂, Alk-His-Thr-OAlk, Alk-Lys-Thr-NR₁R₂, Alk-Lys-Thr-OAlk. When used on the left side of the sequence, “Alk” refers to an N-acyl derivative as defined herein. When used on the right side of the sequence, “OAlk” refers to an ester group attached to the C terminal carbonyl of Thr (e.g., COOAlk). “NR₁R₂” is as defined herein.

In accordance with another aspect of the present invention, dipeptides of the present invention have the following structure: 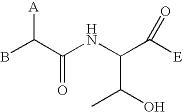
wherein
A=NH₃⁺(CH₂)₄—,

NH₂⁺═C(NH₂)NH—(CH₂)₃— or 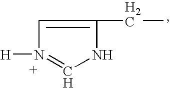
B=—NH₂, —NH₃⁺, —NH-D,
D=an acyl group of 2-22 carbon atoms in length, or biotinyl and
E=—O-Alk, —NR₁R₂, —H, —O⁻, or —OH,
wherein Alk is an alkyl group of 1-24 carbons in length, and R₁ and R₂ are independently H or an alkyl group of 1-12 carbons in length. In a particularly preferred embodiment, B=—NH-D. Note that the molecules of A (Lys, Arg and His respectively) are shown in their respective charged states at pH 6.0. It is understood that they may be present in an uncharged state as well and the representation of A above is meant to include both.

The dipeptides in accordance with the present invention, when provided in personal care compositions, are preferably provided in an amount which is safe and effective to treat at least one sign of an undesired keratinous tissue (e.g., skin, hair, or nail) condition. The phrase “to treat at least one undesired keratinous tissue (e.g., skin, hair, or nail) condition” as used herein means that the dipeptide provides an objectively measurable increase in its effect on some aspect of the keratinous tissue (e.g., skin, hair, or nail) condition when used topically in an effective amount. This can be, for example, a greater reduction in wrinkles, increased potency, the ability to stimulate or inhibit at least one biochemical process within the skin, hair, or nails to a greater degree, and the like. Generally, this is determined based on comparison to a control.

The dipeptide is preferably included in an amount of from about 1×10⁻⁶% to about 10%, more preferably from about 1×10⁻⁶% to about 0.1%, and even more preferably from about 1×10-5% to about 0.01%, by weight of the personal care composition.

Reference to a “dipeptide” in accordance with the present invention means a dipeptide whose C terminal amino acid is Thr. These include, unless the context specifies otherwise, N-acyl derivatives thereof, as well as C terminal derivatives such as esters, acid halides, and nitrogen containing compounds as discussed herein.

The N-acyl derivatives are groups attached to the N terminal amino acid in place of a hydrogen and can include alkyl chains of carbon lengths of between 2 and 22 carbons. These can be linear or branched, substituted or unsubstituted, saturated or unsaturated, hydroxylated or not, containing sulfur or not. N-Acyl may also represent a biotinyl group. Similarly, the threonine may be in the form of a C terminal derivative including, for example, an acid, an ester with an alkyl chain having a carbon length of between 1 and 24 carbons (“Oalk”), preferably 1 to 3 carbons or 14 to 18 carbons. These can be linear or branched, substituted or unsubstituted, saturated or unsaturated, hydroxylated or not, containing sulfur or not. The C terminal derivative may also be NR1R2, in which R1 and R2 are independent of each other H or an alkyl chain of carbon length of between 1 and 12 carbons. These can be linear or branched, substituted or unsubstituted, saturated or unsaturated, hydroxylated or not, containing sulfur or not. Preferably, the acyl derivative attached to the N terminal amino acid is a palmitoyl group and most preferably, the C terminal amino acid is in the form of an acid.

All terms such as “skin aging”, “signs of skin aging”, and the like are used in the sense in which they are generally and widely used in the art of developing, testing and marketing personal care products. “Wrinkles” means furrows in the otherwise smooth surface of the facial skin, visible to the naked eye, generally in the average depth of 50 to more than 200 μm and essentially appearing with progressive age.

The term “amino acid” as employed herein includes and encompasses all of the naturally occurring and synthetic amino acids, either in the D- or L-configuration if optically active. The term “dipeptide” means a molecule comprising two amino acids as defined herein.

In order to enhance the bioavailability and cutaneous and/or epithelial barrier crossing of those peptides, their lipophilicity or lipophilic character can be increased either by acylation of the N-terminal NH₂ group of the peptide, by esterification of the carboxyl group with an alcohol, linear or branched, saturated or unsaturated, hydroxylated or not, or both.

In preferred methods of implementation of the invention, N-acyl groups used are lauroyl (C₁₂) or myristoyl (C₁₄) or palmitoyl (C₁₆) or stearoyl (C₁₈) or oleoyl (C_{18:1}) or arachidic (C₂₀) or linoleoyl (C_{18:2}). Biotinyl groups (biotin or derivatives) are also preferred. In a particularly preferred embodiment, the N terminal group is either H or Palmitoyl.

B. Other Peptides

The compositions of the present invention can comprise a peptide in addition to the peptide of the present invention. Suitable peptides can include, but are not limited to, di-, tri-, tetra-, penta-, and hexa-peptides and derivatives thereof. In one embodiment, the compositions comprise from about 1×10−7% to about 20%, more preferably from about 1×10−6% to about 10%, even more preferably from about 1×10−5% to about 5%, by weight of optional peptide.

In certain embodiments, the peptides contain at least one basic amino acid (e.g., histidine, lysine, arginine). One preferred peptides are the dipeptide carnosine (beta-ala-his), the tripeptide gly-his-lys, the tripeptide his-gly-gly, the tripeptide gly-gly-his, the tripeptide gly-his-gly, the tetrapeptide gly-gln-pro-arg, the pentapeptide lys-thr-thr-lys-ser, lipophilic derivatives of peptides, and metal complexes of the aforementioned (e.g., copper complex of the tripeptide his-gly-gly (also known as Iamin)). Other suitable peptides include Peptide CK (arg-lys-arg); Peptide CK+ (ac-arg-lys-arg-NH2); and Peptide E, arg-ser-arg-lys. A preferred commercially available tripeptide derivative-containing composition is Biopeptide CL®, which contains 100 ppm of palmitoyl-gly-his-lys and is commercially available from Sederma, France. Suitable pentapeptides are disclosed in U.S. Pat. No. 6,492,326, issued Dec. 10, 2002, to Robinson et al. A preferred commercially available pentapeptide derivative-containing composition is Matrixyl®, which contains 100 ppm of palmitoyl-lys-thr-thr-lys-ser and is commercially available from Sederma, France.

Peptide derivatives useful herein include lipophilic derivatives, preferably palmitoyl derivatives. Preferably, the peptide is selected from palmitoyl-lys-thr-thr-lys-ser, palmitoyl-gly-his-lys, their derivatives, and combinations thereof.

C. Hexamidine

The compositions of the present invention can include hexamidine compounds, its salts, and derivatives. Suitable hexamidine compounds useful in the present invention include those compositions that correspond to those of the following chemical structure: 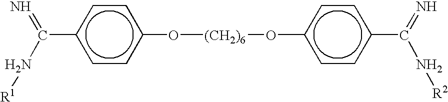
wherein R¹ and R² are organic acids (e.g., sulfonic acids, etc.).

In one embodiment, the hexamidine comprises from about 0.0001% to about 25%, more preferably from about 0.001% to about 10%, more preferably from about 0.01% to about 5%, and even more preferably from about 0.02% to about 2.5% by weight of the composition.

As used herein, hexamidine derivatives include any isomers and tautomers of hexamidine compounds including but not limited to organic acids and mineral acids, for example sulfonic acid, carboxylic acid, etc. Preferably, the hexamidine compounds include hexamidine diisethionate, commercially available as Eleastab® HP100 from Laboratoires Serobiologiques.

D. Vitamin B₃ Compounds

The compositions of the present invention can include a vitamin B3 compound. Vitamin B3 compounds are particularly useful for regulating skin conditions, as described in U.S. Pat. No. 5,939,082. In one embodiment, the composition comprises from about 0.001% to about 50%, more preferably from about 0.01% to about 20%, even more preferably from about 0.05% to about 10%, and still more preferably from about 0.1% to about 7%, even more preferably from about 0.5% to about 5%, by weight of the composition, of the vitamin B3 compound.

As used herein, “vitamin B3 compound” means a compound having the formula: 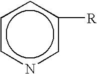
wherein R is —CONH₂ (i.e., niacinamide), —COOH (i.e., nicotinic acid) or —CH2OH (i.e., nicotinyl alcohol); derivatives thereof; and salts of any of the foregoing.

Exemplary derivatives of the foregoing vitamin B3 compounds include nicotinic acid esters, including non-vasodilating esters of nicotinic acid (e.g, tocopherol nicotinate, myristyl nicotinate), nicotinyl amino acids, nicotinyl alcohol esters of carboxylic acids, nicotinic acid N-oxide and niacinamide N-oxide.

E. Sugar Amines (Amino Sugars)

The compositions of the present invention can comprise a sugar amine, which is also known as amino sugar. Sugar amine compounds useful in the present invention can include those described in PCT Publication WO 02/076423 and U.S. Pat. No. 6,159,485.

In one embodiment, the composition comprises from about 0.01% to about 15%, more preferably from about 0.1% to about 10%, and even more preferably from about 0.5% to about 5% by weight of the composition, of sugar amine.

Sugar amines can be synthetic or natural in origin and can be used as pure compounds or mixtures of compounds (e.g., extracts from natural sources or mixtures of synthetic materials). For example, glucosamine is generally found in many shellfish and can also be derived from fungal sources. As used herein, “sugar amine” includes isomers and tautomers of such and its salts (e.g., HCl salt) and is commercially available from Sigma Chemical Co. Examples of sugar amines that are useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine.

F. Other Optional Components

The compositions of the present invention can comprise one or more suitable desired optional components. For example, the composition can optionally include other active or inactive ingredients. For instance, such materials can be selected from the group consisting of, sodium dehydroacetate, dehydroacetic acid and its salts, phytosterols, soy derivatives (e.g., equol and other isoflavones), phytantriol, farnesol, bisabolol, salicylic acid compounds, dialkanoyl hydroxyproline compounds, flavonoids, N-acyl amino acid compounds, retinoids (e.g., retinyl propionate), water-soluble vitamins, ascorbates (e.g., vitamin C, ascorbic acid, ascorbyl glucoside, ascorbyl palmitate, magnesium ascorbyl phosphate, sodium ascorbyl phosphate), particulate materials, sunscreen actives, anti-cellulite agents, butylated hydroxytoluene, butylated hydroxyanisole, their derivatives, and combinations thereof. Other examples of optional ingredients can include cationic polymers, conditioning agents (hydrocarbon oils, fatty esters, silicones), anti dandruff agents, antiseborrheic agents, antipsoriasis agents, suspending agents, viscosity modifiers, dyes, nonvolatile solvents or diluents (water soluble and insoluble), pearlescent aids, foam boosters, surfactants, nonionic cosurfactants, pediculocides, pH adjusting agents, perfumes, preservatives, chelants, chelating agents, proteins, UV absorbers, pigments, other amino acids, and other vitamins.

For instance, the compositions of the present invention may comprise one or more vitamins and/or amino acids such as: water soluble vitamins such as vitamin B1, B2, B6, B12, C, pantothenic acid, pantothenyl ethyl ether, panthenol, biotin, and their derivatives, water soluble amino acids such as asparagine, alanine, indole, glutamic acid and their salts, water insoluble vitamins such as vitamin A, D, E, and their derivatives, water insoluble amino acids such as tyrosine, tryptamine, and their salts.

The compositions of the present invention may also contain one or more pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names. The compositions of the present invention may also contain antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

Any other suitable optional component can also be included in the personal care composition of the present invention, such as those ingredients that are conventionally used in given product types. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein. Examples of these ingredient classes include, but are not limited to: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antibacterial agents, antifungal agents, antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, plant derivatives, plant extracts, plant tissue extracts, plant seed extracts, plant oils, botanicals, botanical extracts, preservatives, propellants, reducing agents, sebum control agents, sequestrants, skin bleaching and lightening agents, (e.g. hydroquinone, kojic acid, ascorbic acid, magnesium ascorbyl phosphate, ascorbyl glucoside, pyridoxine), enzymes, coenzymes, skin-conditioning agents (e.g. humectants and occlusive agents), skin soothing and/or healing agents and derivatives (e.g. panthenol, and derivatives such as ethyl panthenol, aloe vera, pantothenic acid and its derivatives, allantoin, bisabolol, and dipotassium glycyrrhizinate), skin treating agents (e.g. vitamin D compounds, mono-, di-, and tri-terpenoids, beta-ionol, cedrol), thickeners, and vitamins and derivatives thereof. Further optional components suitable for the present personal care composition are described in U.S. Publication No. 2007/0020220A1.

G. Dermatologically Acceptable Carrier

The topical compositions of the present invention can also comprise a dermatologically acceptable carrier for the composition. In one embodiment, the carrier is present at a level of from about 50% to about 99.99%, preferably from about 60% to about 99.9%, more preferably from about 70% to about 98%, and even more preferably from about 80% to about 95%, by weight of the composition.

The carrier can be in a wide variety of forms. Non-limiting examples include simple solutions (water or oil based), emulsions, and solid forms (gels, sticks). For example, emulsion carriers can include, but are not limited to, oil-in-water, water-in-oil, water-in-silicone, water-in-oil-in-water, and oil-in-water-in-silicone emulsions.

Depending upon the desired product form, preferred carriers can comprise an emulsion such as oil-in-water emulsions (e.g., silicone in water) and water-in-oil emulsions, (e.g., water-in-silicone emulsions). As will be understood by the skilled artisan, a given component will distribute primarily into either the water or oil phase, depending on the water solubility/dispensability of the component in the composition. In one embodiment, oil-in-water emulsions are especially preferred.

Emulsions according to the present invention can contain an aqueous phase and a lipid or oil. Lipids and oils may be derived from animals, plants, or petroleum and may be natural or synthetic (i.e., man-made). Preferred emulsions can also contain a humectant, such as glycerin. Emulsions can further comprise from about 0.1% to about 10%, more preferably from about 0.2% to about 5%, of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The compositions of the present invention can be in the form of pourable liquids (under ambient conditions). The compositions can therefore comprise an aqueous carrier, which is typically present at a level of from about 20% to about 95%, preferably from about 60% to about 85%. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

### II. COMPOSITION PREPARATION

The compositions useful for the methods of the present invention are generally prepared by conventional methods such as are known in the art of making topical compositions. Such methods typically can involve mixing of the ingredients in one or more steps to a relatively uniform state, with or without heating, cooling, application of vacuum, and the like.

### III. METHODS FOR TREATING KERATINOUS TISSUE CONDITION

The compositions of the present invention can be useful for treating a number of mammalian keratinous tissue conditions. Such treatment of keratinous tissue conditions can include prophylactic and therapeutic regulation, including regulating the cosmetic appearance of the mammalian keratinous tissue. More specifically, such treatment methods can be directed to, but are not limited to, preventing, retarding, and/or treating uneven skin tone, reducing the size of pores in mammalian skin, regulating oily/shiny appearance of mammalian skin, thickening keratinous tissue (i.e., building the epidermis and/or dermis and/or subcutaneous layers of the skin and where applicable the keratinous layers of the nail and hair shaft), preventing, retarding, and/or treating uneven skin tone by acting as a lightening or pigmentation reduction cosmetic agent, preventing, retarding, and/or treating atrophy of mammalian skin, softening and/or smoothing lips, hair and nails of a mammal, preventing, retarding, and/or treating itch of mammalian skin, preventing, retarding, and/or treating the appearance of dark under-eye circles and/or puffy eyes, preventing, retarding, and/or treating sallowness of mammalian skin, preventing, retarding, and/or treating sagging (i.e., glycation) of mammalian skin, preventing and/or retarding tanning of mammalian skin, desquamating, exfoliating, and/or increasing turnover in mammalian skin, preventing, retarding, and/or treating hyperpigrnentation such as post-inflammatory hyperpigmentation, preventing, retarding, and/or treating the appearance of spider vessels and/or red blotchiness on mammalian skin, preventing, retarding, and/or treating fine lines and wrinkles of mammalian skin, preventing, retarding, and/or treating skin dryness (i.e., roughness, scaling, flaking) and preventing, retarding, and/or treating the appearance of cellulite in mammalian skin. In a preferred embodiment, the composition is used to treat the signs of aging; in one aspect, the composition is used to regulate the signs of aging; in another aspect, the composition is used to reduce or decrease the signs of aging; in yet another aspect the composition is used to prevent the signs of aging in keratinous tissue (e.g., skin, hair, or nails).

For instance, the present invention can be useful for therapeutically regulating visible and/or tactile discontinuities in mammalian keratinous tissue, including discontinuities in skin texture and color. For example, the apparent diameter of pores can be decreased, the apparent height of tissue immediately proximate to pore openings can approach that of the interadnexal skin, the skin tone/color can become more uniform, and/or the length, depth, and/or other dimension of lines and/or wrinkles can be decreased.

Furthermore, compositions of the present invention can also be useful for cleansing (e.g, hair, body, facial), improving keratinous tissue feel (wet & dry) such as for hair (e.g., improving appearance/look, detangling, shine, gloss, decrease coefficient of friction, increase smoothness, color retention, decrease split ends, prevent hair breakage, prevent environmental damage such as sunlight damage, smoke damage, and damage from pollutants such as nitrogen oxides, sulfur oxides, ozone, and metals such as lead), odor control, oil control, conditioning, hair volume control, hair growth, and hair growth inhibition.

Regulating keratinous tissue conditions can involve topically applying to the keratinous tissue a safe and effective amount of a composition of the present invention. The amount of the composition that is applied, the frequency of application, and the period of use will vary widely depending upon the level of components of a given composition and the level of regulation desired, e.g., in view of the level of keratinous tissue damage present or expected to occur.

In one embodiment, the composition is chronically applied to the skin, e.g. topically. By “chronic application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic applications continue throughout the subject's lifetime. Typically applications would be on the order of about once per day over such extended periods, however, application rates can vary, and can include from about once per week up to about three times per day or more.

A wide range of quantities of the compositions of the present invention can be employed to provide a keratinous tissue appearance and/or feel benefit when applied topically. For example, quantities of the present compositions, which are typically applied per application are, in mg composition/cm² keratinous tissue, from about 0.1 mg/cm² to about 20 mg/cm². A particularly useful application amount is about 0.5 mg/cm² to about 10 mg/cm².

Treating keratinous tissue condition can be practiced, for example, by applying a composition in the form of a skin lotion, clear lotion, milky lotion, cream, gel, foam, ointment, paste, emulsion, spray, conditioner, tonic, cosmetic, lipstick, foundation, nail polish, after-shave, or the like which is intended to be left on the skin or other keratinous tissue for some aesthetic, prophylactic, therapeutic or other benefit (i.e., a “leave-on” composition). After applying the composition to the keratinous tissue (e.g., skin), it is preferably left on for a period of at least about 15 minutes, more preferably at least about 30 minutes, even more preferably at least about 1 hour, even more preferably for at least several hours, e.g., up to about 12 hours. Any part of the external portion of the face, hair, and/or nails can be treated, (e.g., face, lips, under-eye area, eyelids, scalp, neck, torso, arms, hands, legs, feet, fingernails, toenails, scalp hair, eyelashes, eyebrows, etc.) The application of the present compositions may be done using the palms of the hands and/or fingers or a device or implement (e.g., a cotton ball, swab, pad, applicator pen, spray applicator, etc.)

Another approach to ensure a continuous exposure of the keratinous tissue to at least a minimum level of the composition is to apply the compound by use of a patch applied, e.g., to the face. Such an approach is particularly useful for problem skin areas needing more intensive treatment (e.g., facial crows feet area, frown lines, under eye area, upper lip, and the like). The patch can be occlusive, semi-occlusive or non-occlusive, and can be adhesive or non-adhesive. The composition can be contained within the patch or be applied to the skin prior to application of the patch. The patch can also include additional actives such as chemical initiators for exothermic reactions such as those described in PCT application WO 9701313, and in U.S. Pat. Nos. 5,821,250, 5,981,547, and 5,972,957 to Wu, et al. The patch can also contain a source of electrical energy (e.g., a battery) to, for example, increase delivery of the composition and active agents (e.g., iontophoresis). The patch is preferably left on the keratinous tissue for a period of at least about 5 minutes, more preferably at least about 15 minutes, more preferably still at least about 30 minutes, even more preferably at least about 1 hour, even more preferably at night as a form of night therapy.

### IV. EXAMPLES

The following are non-limiting examples of compositions of the present invention. The examples are given solely for the purpose of illustration and are not to be construed as limitations of the present invention, as many variations thereof are possible without departing from the spirit and scope of the invention, which would be recognized by one of ordinary skill in the art.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

### Examples 1-5

### Moisturizing Oil-in-Water Lotions/Creams

| | | | | |
|---|---|---|---|---|
| 1 | 2 | 3 | 4 | 5 |

Water Phase:

| | | | | | |
|---|---|---|---|---|---|
| Water | Qs | qs | Qs | qs | qs |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| Triethanolamine | — | 0.25 | — | — | — |
| D-panthenol | 0.5 | 0.1 | — | 0.5 | 1.5 |
| Sodium Dehydroacetate | 0.5 | 0.1 | 0.5 | 0.1 | 0.5 |
| Benzyl alcohol | 0.25 | 0.25 | 0.25 | 0.25 | 0.25 |
| GLW75CAP-MP (75% aq. | — | 0.5 | 0.5 | — | — |
| TiO2 dispersion)¹ | | | | | |
| Hexamidine diisethionate | — | 0.1 | — | — | — |
| Palmitoyl-dipeptide² | 0.00055 | 0.00055 | 0.0001 | 0.00055 | 0.00055 |
| N-acetyl glucosamine | 2 | 1 | 2 | 2 | 1 |
| Soy Isoflavone | 0.5 | — | — | — | — |

Oil Phase:

| | | | | | |
|---|---|---|---|---|---|
| Salicylic Acid | — | — | 1.5 | — | — |
| Isohexadecane | 3 | 3 | 3 | 4 | 3 |
| PPG15 Stearyl Ether | — | — | 4 | — | — |
| Isopropyl Isostearate | 1 | 0.5 | 1.3 | 1.5 | 1.3 |
| Sucrose polyester | 0.7 | — | 0.7 | 1 | 0.7 |
| Dipalmitoylhydroxyproline | — | — | — | 1.0 | — |
| Undecylenoyl Phenylalanine | — | 0.5 | — | — | — |
| Phytosterol | — | — | 0.5 | — | 1.0 |
| Cetyl alcohol | 0.4 | 0.3 | 0.4 | 0.5 | 0.4 |
| Stearyl alcohol | 0.5 | 0.35 | 0.5 | 0.6 | 0.5 |
| Behenyl alcohol | 0.4 | 0.3 | 0.4 | 0.5 | 0.4 |
| PEG-100 stearate | 0.1 | 0.1 | 0.1 | 0.2 | 0.1 |
| Cetearyl glucoside | 0.1 | 0.1 | 0.1 | 0.25 | 0.1 |

Thickener:

| | | | | | |
|---|---|---|---|---|---|
| Polyacrylamide/C13-14 | 1.5 | — | 2 | 2.5 | 2 |
| isoparaffin/laureth-7 | | | | | |
| Sodium acrylate/sodium | — | 3 | — | — | — |
| acryloyldimethyl taurate | | | | | |
| copolymer/isohexadecane/ | | | | | |
| polysorbate 80 | | | | | |

Additional Ingredients:

| | | | | | |
|---|---|---|---|---|---|
| Dimethicone/dimethiconol | — | 1 | 2 | 0.5 | 2 |
| Polymethylsilsequioxane | — | — | 0.25 | — | 1 |
| Nylon-12 | — | 0.5 | — | — | — |
| Prestige Silk Violet³ | — | — | — | — | 1 |
| Timiron Splendid Red⁴ | — | 1.0 | — | 2 | — |

¹Available from Kobo products
²Palmitoyl-lysine-threonine available from Sederma
³Titanium dioxide coated mica violet interference pigment available from Eckart
⁴Silica and titanium dioxide coated mica red interference pigment available from Rona

In a suitable vessel, combine the water phase ingredients and heat to 75° C. In a separate suitable vessel, combine the oil phase ingredients and heat to 75° C. Next, add the oil phase to the water phase and mill the resulting emulsion (e.g., with a Tekmar T-25). Then, add the thickener to the emulsion and cool the emulsion to 45° C. while stirring. At 45° C., add the remaining ingredients. Cool the product and stir to 30° C. and pour into suitable containers.

### Examples 6-12

### Moisturizing Silicone-in-Water Serums/Lotions

| | | | | | | |
|---|---|---|---|---|---|---|
| 6 | 7 | 8 | 9 | 10 | 11 | 12 |

Water Phase:

| | | | | | | | |
|---|---|---|---|---|---|---|---|
| Water | Qs | Qs | Qs | qs | Qs | qs | Qs |
| Glycerin | 3 | 3 | 5 | 7 | 10 | 15 | 10 |
| Disodium EDTA | 0.1 | 0.1 | 0.1 | 0.05 | 0.1 | 0.1 | 0.1 |
| Niacinamide | 2 | 2 | 0.5 | — | 3 | 5 | 3 |
| Sodium Dehydroacetate | 0.5 | — | 0.1 | — | 0.1 | 0.5 | 0.1 |
| D-panthenol | 0.5 | 0.5 | 0.1 | | 0.5 | 1.5 | 0.5 |
| GLW75CAP-MP (75% aq. | — | — | 0.4 | — | — | — | 0.4 |
| TiO2 dispersion)¹ | | | | | | | |
| Ascorbyl Glucoside | — | — | — | — | — | — | 1 |
| Palmitoyl dipeptide² | 0.00055 | 0.00055 | 0.00055 | 0.00055 | 0.00055 | 0.00055 | 0.00055 |
| Palmitoyl pentapeptide³ | — | 0.0003 | — | — | — | — | — |
| Carnosine | — | 0.1 | — | — | — | — | — |
| Soy Isoflavone | — | — | 1 | — | — | — | — |
| N-acetyl glucosamine | 2 | — | — | 2 | — | 5 | — |

Silicone/Oil Phase:

| | | | | | | | |
|---|---|---|---|---|---|---|---|
| Cyclomethicone D5 | 10 | 10 | 5 | 5 | 10 | 7.5 | 10 |
| Dow Corning 9040 Silicone | — | — | 10 | 5 | 5 | 7.5 | 5 |
| elastomer⁴ | | | | | | | |
| KSG-15AP silicone | 5 | 5 | — | 5 | 5 | 7.5 | 5 |
| Elastomer⁵ | | | | | | | |
| Dimethione/dimethiconol | — | — | 2 | 2 | 1 | 2 | 1 |
| Dimethicone 50 csk | 1 | 1 | — | — | — | — | — |
| Salicylic Acid | — | — | — | 1.5 | — | — | — |
| Phytosterol | — | — | — | — | 1.0 | — | 0.1 |
| PPG-15 Stearyl Ether | — | — | — | 4 | 4 | — | — |
| Dehydroacetic acid | — | — | — | 0.5 | — | — | — |
| Undecylenoyl Phenylalanine | — | — | — | 0.5 | — | — | — |
| BHT | — | — | 0.5 | — | — | — | — |
| Vitamin E Acetate | — | — | 0.5 | 0.1 | 0.1 | — | 0.1 |

Thickener:

| | | | | | | | |
|---|---|---|---|---|---|---|---|
| Polyacrylamide/C13-14 | 2.5 | 2.5 | 2.5 | — | — | — | 3 |
| isoparaffin/laureth-7 | | | | | | | |
| Sodiumacrylate/sodium | — | — | — | — | 3 | — | — |
| acryloyl dimethyl taurate | | | | | | | |
| copolymer/isohexadecane/ | | | | | | | |
| polysorbate 80 | | | | | | | |
| Acrylates/C10-30 alkyl | — | — | — | 0.6 | — | 0.5 | — |
| acrylates crosspolymer | | | | | | | |

Undecylenoyl Phenylalanine Premix

| | | | | | | | |
|---|---|---|---|---|---|---|---|
| Undecylenoyl Phenylalanine | — | — | — | — | — | 1 | — |
| Water | — | — | — | — | — | 24 | — |
| Triethanolamine | — | — | — | — | — | 0.5 | — |

Dipalmitoyl Hydroxy-Proline Premix:

| | | | | | | | |
|---|---|---|---|---|---|---|---|
| Water | — | — | — | — | — | — | 4.4 |
| Triethanolamine | — | — | — | — | — | — | 0.1 |
| Dipalmitoylhyroxy proline | — | — | — | — | — | — | 1.0 |

Additional Ingredients:

| | | | | | | |
|---|---|---|---|---|---|---|
| Triethanolamine | — | — | — | — | 0.6 | — |
| Polymethylsilsequioxane | 0.5 | 0.5 | 1.0 | 1 | 1 | 0.5 |
| Polyethylene | — | 0.5 | 0.5 | 1.0 | — | — |
| Flamenco Summit | — | — | 1.0 | — | — | — |
| Green G30D⁶ | | | | | | |
| Silca | — | — | 1 | 0.5 | — | — |
| Prestige Silk Red⁷ | — | — | — | 1.0 | 1.0 | 1.0 |

¹GLW75CAP-MP, 75% aqueous titanium dioxide dispersion from Kobo
²Palmitoyl-lysine-threonine available from Sederma
³Palmitoyl-lys-thr-thr-lys-ser available from Sederma
⁴A silicone elastomer dispersion from Dow Corning Corp
⁵A silicone elastomer dispersion from Shin Etsu,
⁶Titanium dioxide and tin oxide coated mica green interference pigment from Engelhard
⁷Titanium dioxide coated mica red interference pigment from Eckart

In a suitable vessel, combine the water phase ingredients and mix until uniform. In a separate suitable container, combine the silicone/oil phase ingredients and mix until uniform. Separately, prepare the dipalmitoyl hydroxyproline premix and/or undecylenoyl phenylalanine premix by combining the premix ingredients in a suitable container, heat to about 70° C. while stirring, and cool to room temperature while stirring. Add half the thickener and then the silicone/oil phase to the water phase and mill the resulting emulsion (e.g., with a Tekmar T-25). Add the remainder of the thickener, the dipalmitoyl hydroxyproline premix and/or undecylenoyl phenylalanine premix, and then the remaining ingredients to the emulsion while stirring. Once the composition is uniform, pour the product into suitable containers.

### Examples 13-19

### Moisturizing Water-in-Silicone Creams/Lotions

| Component | | | | | | |
|---|---|---|---|---|---|---|
| 13 | 14 | 15 | 16 | 17 | 18 | 19 |

Phase A

| | | | | | | | |
|---|---|---|---|---|---|---|---|
| Water | qs | qs | qs | qs | qs | qs | qs |
| Allantoin | 0.2 | 0.2 | 0.2 | 0.2 | 0.2 | 0.2 | 0.2 |
| disodium EDTA | 0.1 | 0.1 | 0.1 | 0.1 | 0.1 | 0.1 | 0.1 |
| ethyl paraben | 0.2 | 0.2 | 0.2 | 0.2 | 0.2 | 0.2 | 0.2 |
| propyl paraben | 0.1 | 0.1 | 0.1 | 0.1 | 0.1 | 0.1 | 0.1 |
| Caffeine | — | 1 | — | — | — | — | 1 |
| BHT | — | 0.1 | — | — | 0.015 | — | — |
| Dexpanthenol | 1 | 0.5 | 1 | 1 | 1 | 1 | 1 |
| Glycerin | 7.5 | 10 | 15 | 15 | 7.5 | 5 | 15 |
| hexamidine isethionate | — | — | 0.1 | 0.1 | 0.5 | — | — |
| Niacinamide | 2 | — | 4 | — | 2 | 3.5 | 5 |
| Palmitoyl-dipeptide¹ | 0.00055 | 0.00055 | 0.00055 | 0.00055 | 0.00055 | 0.00055 | 0.00055 |
| Palmitoyl-pentapeptide² | — | — | 0.0003 | — | — | — | — |
| Phenylbenzimidazole | — | — | — | — | — | 1 | — |
| sulfonic acid | | | | | | | |
| Sodium Dehydroacetate | 0.5 | — | — | — | 0.1 | 0.5 | 0.5 |
| benzyl alcohol | 0.25 | 0.25 | 0.25 | 0.25 | 0.25 | 0.25 | 0.25 |
| Triethanolamine | — | — | — | — | — | 0.6 | — |
| green tea extract | 1 | 1 | 1 | 1 | 1 | 1 | 1 |
| Soy Isoflavone | — | 0.5 | — | — | — | — | — |
| N-acetyl glucosamine | 5 | — | 2 | 2 | 5 | 2 | — |
| Sodium metabisulfite | 0.1 | 0.1 | 0.1 | 0.1 | 0.1 | 0.1 | 0.1 |

Phase B

| | | | | | | | |
|---|---|---|---|---|---|---|---|
| Cyclopentasiloxane | 15 | 15 | 18 | 18 | 15 | 15 | 18 |
| Titanium dioxide | 0.5 | 0.5 | 0.75 | 0.75 | 0.5 | 0.5 | 0.75 |

Phase C

| | | | | | | | |
|---|---|---|---|---|---|---|---|
| C12-C15 alkyl benzoate | — | — | — | — | 1.5 | 1.5 | — |
| Vitamin E acetate | 0.5 | — | 1 | 1 | 0.5 | 0.5 | 1 |
| retinyl propionate | 0.3 | — | — | — | 0.2 | 0.2 | — |
| Undecylenoyl Phenylalanine | — | — | — | 0.5 | — | — | — |
| Dipalmitoyl hydroxyproline | — | 1 | — | — | — | — | — |
| Salicylic Acid | — | 1.5 | — | 1.5 | — | — | — |
| PPG-15 Stearyl Ether | 4 | 4 | — | 4 | — | — | — |
| Dehydroacetic Acid | — | 0.5 | — | 0.1 | — | — | — |
| Phytosterol | 1 | 0.5 | — | — | — | — | — |

Phase D

| | | | | | | | |
|---|---|---|---|---|---|---|---|
| KSG-21 silicone elastomer³ | 4 | 4 | 5 | 5 | 4 | 4 | 5 |
| Dow Corning 9040 silicone | 15 | 15 | 12 | 12 | 15 | 15 | 12 |
| elastomer⁴ | | | | | | | |
| Abil EM-97 Dimethicone | 0.5 | — | — | — | 0.5 | 0.5 | — |
| Copolyol⁵ | | | | | | | |
| Polymethylsilsesquioxane | 2.5 | 2.5 | 2 | 2 | 2.5 | 2.5 | 2 |

Undecylenoyl Phenylalanine Premix

| | | | | | | | |
|---|---|---|---|---|---|---|---|
| Undecylenoyl Phenylalanine | — | — | — | — | — | 1 | — |
| Water | — | — | — | — | — | 24 | — |
| Triethanolamine | — | — | — | — | — | 0.5 | — |

Phase E

| | | | | | | | |
|---|---|---|---|---|---|---|---|
| Water | 8.8 | — | — | — | — | — | 8.85 |
| Triethanolamine | 0.2 | — | — | — | — | — | 0.25 |
| Dipalmitoylhyroxyproline | 0.5 | — | — | — | — | — | 1 |

¹Palmitoyl-lysine-threonine available from Sederma
²Palmitoyl-lys-thr-thr-lys-ser available from Sederma
³KSG-21 is an emulsifying silicone elastomer available from Shin Etsu
⁴A silicone elastomer dispersion from Dow Corning Corp
⁵Abil EM-97 available from Goldschmidt Chemical Corporation

In a suitable vessel, blend the Phase A components together with a suitable mixer (e.g., Tekmar model RW20DZM) and mix until all of the components are dissolved. Then, blend the Phase B components together in a suitable vessel and mill using a suitable mill (e.g., Tekmar RW-20) for about 5 minutes. Add the Phase C components to the Phase B mixture with mixing. Then, add the Phase D components to the mixture of Phases B and C and then mix the resulting combination of Phase B, C and D components using a suitable mixer (e.g., Tekmar RW-20) for about 1 hour. If applicable, prepare the undecylenoyl phenylalanine premix and/or Phase E by combining all ingredients, heating the ingredients to 70° C. while stirring, and cooling back to room temperature while stirring. Add the undecylenoyl phenylalanine premix and/or Phase E to Phase A while mixing. Next, slowly add Phase A to the mixture of Phases B, C and D with mixing. Mix the resulting mixture continually until the product is uniform. Mill the resulting product for about 5 minutes using an appropriate mill (e.g., Tekmar T-25).

### Examples 20-24

### Oil in Water Mousse

| | | | | |
|---|---|---|---|---|
| 20 | 21 | 22 | 23 | 24 |

Water Phase:

| | | | | | |
|---|---|---|---|---|---|
| Water | Qs | qs | Qs | qs | qs |
| Glycerin | 3 | 5 | 7 | 10 | 15 |
| Disodium EDTA | 0.1 | 0.1 | 0.05 | 0.1 | 0.1 |
| Methylparaben | 0.1 | 0.1 | 0.1 | 0.1 | 0.1 |
| Niacinamide | 2 | 0.5 | — | 3 | 5 |
| Triethanolamine | — | 0.25 | — | — | — |
| D-panthenol | 0.5 | 0.1 | — | 0.5 | 1.5 |
| Sodium Dehydroacetate | 0.5 | 0.1 | 0.5 | 0.1 | 0.5 |
| Benzyl alcohol | 0.25 | 0.25 | 0.25 | 0.25 | 0.25 |
| GLW75CAP-MP (75% aq. TiO2 | — | 0.5 | 0.5 | — | — |
| dispersion)¹ | | | | | |
| Undecylenoyl Phenylalanine | 1 | — | 0.5 | — | — |
| Hexamidine diisethionate | — | 0.1 | — | — | — |
| Palmitoyl-dipeptide² | 0.00055 | 0.00055 | 0.0001 | 0.00055 | 0.00055 |
| N-acetyl glucosamine | 2 | 1 | 2 | 2 | 1 |
| Soy Isoflavone | 0.5 | — | — | — | — |

Oil Phase:

| | | | | | |
|---|---|---|---|---|---|
| Salicylic Acid | — | — | 1.5 | — | — |
| Isohexadecane | 3 | 3 | 3 | 4 | 3 |
| PPG15 Stearyl Ether | — | — | 4 | — | — |
| Isopropyl Isostearate | 1 | 0.5 | 1.3 | 1.5 | 1.3 |
| Sucrose polyester | 0.7 | — | 0.7 | 1 | 0.7 |
| Undecylenoyl Phenylalanine | — | 0.5 | — | — | — |
| Dipalmitoylhyroxyproline | — | — | — | 1.0 | — |
| Phytosterol | — | — | 0.5 | — | 1.0 |
| Cetyl alcohol | 0.4 | 0.3 | 0:4 | 0.5 | 0.4 |
| Stearyl alcohol | 0.5 | 0.35 | 0.5 | 0.6 | 0.5 |
| Behenyl alcohol | 0.4 | 0.3 | 0.4 | 0.5 | 0.4 |
| PEG-100 stearate | 0.1 | 0.1 | 0.1 | 0.2 | 0.1 |
| Cetearyl glucoside | 0.1 | 0.1 | 0.1 | 0.25 | 0.1 |

Thickener:

| | | | | | |
|---|---|---|---|---|---|
| Polyacrylamide/C13-14 | 1.5 | — | 2 | 2.5 | 2 |
| isoparaffin/laureth-7 | | | | | |
| Sodium acrylate/sodium | — | 3 | — | — | — |
| acryloyldimethyl taurate | | | | | |
| copolymer/isohexadecane/ | | | | | |
| polysorbate 80 | | | | | |

Additional Ingredients:

| | | | | | |
|---|---|---|---|---|---|
| Dimethicone/dimethiconol | — | 1 | 2 | 0.5 | 2 |
| Polymethylsilsequioxane | — | — | 0.25 | — | 1 |
| Nylon-12 | — | 0.5 | — | — | — |
| Prestige Silk Violet³ | — | — | — | — | 1 |
| Timiron Splendid Red⁴ | — | 1.0 | — | 2 | — |

Propellant Phase

| | | | | | |
|---|---|---|---|---|---|
| 152 A HFC Propellant | 3 | 4 | 2 | 3 | 2 |
| A-70 Propellant | 3 | 2 | 4 | 3 | 4 |

¹Available from Kobo products
²Palmitoyl-lysine-threonine available from Sederma
³Titanium dioxide coated mica violet interference pigment available from Eckart
⁴Silica and titanium dioxide coated mica red interference pigment available from Rona

In a suitable vessel, combine the water phase ingredients and heat to 75° C. In a separate suitable vessel, combine the oil phase ingredients and heat to 75° C. Next, add the oil phase to the water phase and mill the resulting emulsion (e.g., with a Tekmar T-25). Add the thickener to the emulsion and cool the emulsion to 45° C. while stirring. At 45° C., add the remaining ingredients. Cool the product with stirring to 30° C. and pour into suitable containers. Add propellant and product to a suitable aerosol container, and seal the container.

### Examples 25-30

### Silicone in Water Mousse

| | | | | | |
|---|---|---|---|---|---|
| 25 | 26 | 27 | 28 | 29 | 30 |

Water Phase:

| | | | | | | |
|---|---|---|---|---|---|---|
| Water | Qs | Qs | qs | Qs | qs | qs |
| Glycerin | 3 | 5 | 7 | 10 | 15 | 10 |
| Disodium EDTA | 0.1 | 0.1 | 0.05 | 0.1 | 0.1 | 0.1 |
| Niacinamide | 2 | 0.5 | — | 3 | 5 | 3 |
| Sodium Dehydroacetate | 0.5 | 0.1 | — | 0.1 | 0.5 | 0.1 |
| D-panthenol | 0.5 | 0.1 | — | 0.5 | 1.5 | 0.5 |
| GLW75CAP-MP (75% aq. TiO2 | — | 0.4 | — | — | — | 0.4 |
| dispersion)¹ | | | | | | |
| Ascorbyl Glucoside | — | — | — | — | — | 1 |
| Palmitoyl dipeptide² | 0.00055 | 0.00055 | 0.00055 | 0.00055 | 0.00055 | 0.00055 |
| Soy Isoflavone | — | 1 | — | — | — | — |
| N-acetyl glucosamine | 2 | — | 2 | — | 5 | — |

Silicone/Oil Phase:

| | | | | | | |
|---|---|---|---|---|---|---|
| Cyclomethicone D5 | 10 | 5 | 5 | 10 | 7.5 | 10 |
| Dow Corning 9040 | — | 10 | 5 | 5 | 7.5 | 5 |
| Silicone elastomer³ | | | | | | |
| KSG-15AP silicone | 5 | — | 5 | 5 | 7.5 | 5 |
| Elastomer⁴ | | | | | | |
| Dimethione/Dimethiconol | — | 2 | 2 | 1 | 2 | 1 |
| Dimethicone 50 csk | 1 | — | — | — | — | — |
| Salicylic Acid | — | — | 1.5 | — | — | — |
| Phytosterol | — | — | — | 1.0 | — | 0.1 |
| PPG-15 Stearyl Ether | — | — | 4 | 4 | — | — |
| Dehydroacetic acid | — | — | 0.5 | — | — | — |
| Undecylenoyl Phenylalanine | — | — | 0.5 | — | — | — |
| BHT | — | 0.5 | — | — | — | — |
| Vitamin E Acetate | — | 0.5 | 0.1 | 0.1 | — | 0.1 |

Thickener:

| | | | | | | |
|---|---|---|---|---|---|---|
| Polyacrylamide/C13-14 | 2.5 | 2.5 | — | — | — | 3 |
| isoparaffin/laureth-7 | | | | | | |
| Sodiumacrylate/Sodium | — | — | — | 3 | — | — |
| acryloyldimethyl taurate | | | | | | |
| copolymer/isohexadecane/ | | | | | | |
| polysorbate 80 | | | | | | |
| Acrylates/C10-30 alkyl | — | — | 0.6 | — | 0.5 | — |
| acrylates crosspolymer | | | | | | |

Undecylenoyl Phenylalanine/Dipalmitoyl
Hydroxyproline Premix

| | | | | | | |
|---|---|---|---|---|---|---|
| Undecylenoyl Phenylalanine | — | — | — | — | 1 | — |
| Water | — | — | — | — | 24 | 9 |
| Triethanolamine | — | — | — | — | 0.5 | 0.2 |
| Dipalmitoylhyroxyproline | — | — | — | — | — | 1.0 |

Additional Ingredients:

| | | | | | | |
|---|---|---|---|---|---|---|
| Triethanolamine | — | — | — | — | 0.6 | — |
| Polymethyl Silsequioxane | 0.5 | 0.5 | 1.0 | 1 | 1 | 0.5 |
| Polyethylene | — | 0.5 | 0.5 | 1.0 | — | — |
| Flamenco Summit | — | — | 1.0 | — | — | — |
| Green G30D⁵ | | | | | | |
| Silica | — | — | 1 | 0.5 | — | — |
| Prestige Silk Red⁶ | — | — | — | 1.0 | 1.0 | 1.0 |

Propellant Phase

| | | | | | | |
|---|---|---|---|---|---|---|
| 152A HFCPropellant | 3 | 2 | 4 | 1 | 5 | 3 |
| A-70 Propellant | 3 | 4 | 2 | 5 | 1 | 3 |

¹GLW75CAP-MP, 75% aqueous titanium dioxide dispersion from Kobo
²Palmitoyl-lysine-threonine available from Sederma
³A silicone elastomer dispersion from Dow Corning Corp
⁴A silicone elastomer dispersion from Shin Etsu,
⁵Titanium dioxide and tin oxide coated mica green interference pigment from Engelhard
⁶Titanium dioxide coated mica red interference pigment from Eckart

In a suitable vessel, combine the water phase ingredients and mix until uniform. In a separate suitable container, combine the silicone/oil phase ingredients and mix until uniform. Separately, prepare the undecylenoyl phenylalanine and/or dipalmitoyl hydroxyproline premix by combining the premix ingredients in a suitable container, heat to about 70° C. while stirring, and cool to room temperature while stirring. Add half the thickener and then the silicone/oil phase to the water phase and mill the resulting emulsion (e.g., with a Tekmar T-25). Add the remainder of the thickener, the undecylenoyl phenylalanine and/or dipalmitoyl hydroxyproline premix, and then the remaining ingredients to the emulsion while stirring. Once the composition is uniform, pour the product into suitable containers. Add the product and propellant into an aerosol container. Seal the aerosol container.

### Examples 31-36

### Water Based Stick Formulations

| Water Phase: | 31 | 32 | 33 | 34 | 35 | 36 |
|---|---|---|---|---|---|---|
| Water | Qs | qs | qs | qs | qs | Qs |
| Palmitoyl dipeptide¹ | 0.00055 | 0.00055 | 0.00055 | 0.00055 | 0.00055 | 0.00055 |
| Propylene Glycol | 15 | 25 | 20 | 15 | 25 | 20 |
| Dipropylene Glycol | 50 | 40 | 45 | 50 | 40 | 45 |
| Sodium Stearate | 6 | 6 | 6 | 6 | 6 | 6 |
| Triethanolamine | 0.2 | 0.25 | — | 0.7 | 0.6 | — |
| N-Acetyl-D-Glucosamine | — | 2.0 | 0.5 | — | — | 2.0 |
| Undecyenoyl | — | 0.5 | — | 1 | — | — |
| Phenylalanine | | | | | | |
| Niacinamide | 2 | | 3.5 | | 2 | 3.5 |
| Sodium Dehydroacetate | 0.5 | 0.5 | 0.1 | 0.1 | 0.5 | 1.0 |
| Dipalmitoyl | 1 | — | — | 1 | 0.5 | — |
| Hydroxyproline | | | | | | |

¹Palmitoyl-lysine-threonine available from Sederma

All ingredients are combined into an appropriate size container, heated to 85° C., cooled and poured into stick containers at approximately 65° C.

### Examples 37-42

### Anhydrous Stick Formulations

| Oil Phase: | 37 | 38 | 39 | 40 | 41 | 42 |
|---|---|---|---|---|---|---|
| Isopropyl Isostearate | 5 | 4 | 3 | 5 | 4 | 3 |
| Palmitoyl dipeptide¹ | 0.00055 | 0.00055 | 0.00055 | 0.00055 | 0.00055 | 0.00055 |
| Octylmethoxycinnamate | 5 | 2 | 2 | 5 | 2 | 2 |
| Cyclomethicone | Qs | qs | qs | qs | qs | qs |
| Phenyl trimethicone | 5 | 5 | 5 | 5 | 5 | 5 |
| Stearyl Alcohol | 15 | 17 | 15 | 15 | 17 | 15 |
| Behenyl Alcohol | 1 | 1 | 1 | 1 | 1 | 1 |
| Undecylenoyl Phenyl alanine | — | 0.5 | — | 1.0 | 0.5 | 0.5 |
| Dehydroacetic acid | 0.1 | 0.5 | 0.1 | 0.5 | 0.1 | 1.0 |
| Dipalmitoyl Hydroxyproline | 1 | — | 1.0 | — | 0.5 | — |
| Phytosterol | 1 | 0.5 | — | — | 0.5 | 1 |
| Salicylic Acid | — | — | 0.5 | 1.5 | — | 1.0 |

¹Palmitoyl-lysine-threonine available from Sederma

All ingredients added to an appropriate size container, heated to 75° C. then cooled with stirring until mixture reaches approximately 45° C. The mixture is poured into stick containers.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) a first dipeptide based molecule having a C terminal amino acid and a N terminal amino acid, wherein the C terminal amino acid of said dipeptide based molecule is threonine;
b) a pentapeptide;
c) a second dipeptide other than said first dipeptide based molecule;
d) a vitamin B3 compound; and
e) a dermatologically acceptable carrier.

2. The personal care composition of claim 1 wherein the peptapeptide is palmitoyl-lys-thr-thr-lys-ser.

3. The personal care composition of claim 1 wherein the second dipeptide is caronosine.

4. The personal care composition of claim 1 wherein the vitamin B3 compound is niacinamide and derivatives thereof.

5. The personal care composition of claim 1, wherein the N terminal amino acid of said dipeptide based molecule is a basic amino acid.

6. The personal care composition of claim 5, wherein the N terminal amino acid of said dipeptide based molecule is selected from the group consisting of lysine, arginine, and histidine.

7. The personal care composition of claim 6, wherein the N terminal amino acid of said dipeptide based molecule is lysine.

8. The personal care composition of claim 1, wherein said amino acid comprises an N acyl derivative of said dipeptide based molecule.

9. The personal care composition of claim 8, wherein said N acyl derivative is an amide.

10. The personal care composition according to claim 1, wherein said dipeptide is a derivative selected from the group consisting of His-Thr, Arg-Thr, Lys-Thr, Alk-His-Thr, Alk-Arg-Thr, Alk-Lys-Thr, His-Thr-OAlk, Arg-Thr-OAlk, Lys-Thr-OAlk, His-Thr-N—R1R2, Arg-Thr-NR1R2, Lys-Thr-NR1R2, Alk-His-Thr-OAlk, Alk-Lys-Thr-N—R1R2, Alk-Lys-Thr-OAlk, and combinations thereof; wherein Alk refers to an N-acyl derivative or biotinyl group, OAlk refers to an alkyl group attached to the C terminal carbonyl of said Thr, and R1 and R2 are independently H or an alkyl group of 1-12 carbons in length.

11. A personal care composition comprising:
(a) a first dipeptide, wherein said first dipeptide has the following structure: 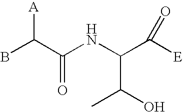
wherein
A=NH₃⁺(CH₂)₄—,
NH₂⁺═C(NH₂)NH—(CH₂)₃— or 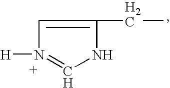
B=—NH₂, —NH₃⁺, —NH-D,
D=an acyl group of 2-22 carbon atoms in length, or biotinyl group and
E=—O-Alk, —NR₁R₂, —H, —O⁻, or —OH,
wherein Alk is an alkyl group of 1-24 carbons in length, and R₁ and R₂ are independently H or an alkyl group of 1-12 carbons in length; and
(b) a pentapeptide;
(c) a second dipeptide other than said first dipeptide based molecule;
(d) a vitamin B3 compound; and
(e) a dermatologically acceptable carrier.

12. The personal care composition of claim 11 wherein the second dipeptide is caronosine.

13. The personal care composition of claim 11 wherein the vitamin B3 compound is niacinamide and derivatives thereof.

14. The personal care composition of claim 11 wherein the peptapeptide is palmitoyl lys-thr-thr-lys-ser.

15. A personal care composition comprising:
(a) a dipeptide based molecule having a C terminal amino acid and a N terminal amino acid, wherein the C terminal amino acid of said dipeptide based molecule is threonine;
(b) a pentapeptide;
(c) a hexamindine compound;
(d) a vitamin B3 compound; and
(e) a dermatologically acceptable carrier.

16. The personal care composition of claim 15 wherein the peptapeptide is palmitoyl-lys-thr-thr-lys-ser.

17. The personal care composition of claim 15 wherein the vitamin B3 compound is niacinamide and derivatives thereof.

18. The personal care composition of claim 15 wherein the N terminal amino acid of said dipeptide based molecule is a basic amino acid.

19. The personal care composition of claim 18, wherein the N terminal amino acid of said dipeptide based molecule is selected from the group consisting of lysine, arginine, and histidine.

20. The personal care composition of claim 19, wherein the N terminal amino acid of said dipeptide based molecule is lysine.

21. The personal care composition of claim 15, wherein said amino acid comprises an N acyl derivative of said dipeptide based molecule.

22. The personal care composition of claim 21, wherein said N acyl derivative is an amide.

23. The personal care composition according to claim 15, wherein said dipeptide is a derivative selected from the group consisting of His-Thr, Arg-Thr, Lys-Thr, Alk-His-Thr, Alk-Arg-Thr, Alk-Lys-Thr, His-Thr-OAlk, Arg-Thr-OAlk, Lys-Thr-OAlk, His-Thr-N—R1R2, Arg-Thr-NR1R2, Lys-Thr-NR1R2, Alk-His-Thr-OAlk, Alk-Lys-Thr-N—R1R2, Alk-Lys-Thr-OAlk, and combinations thereof; wherein Alk refers to an N-acyl derivative or biotinyl group, OAlk refers to an alkyl group attached to the C terminal carbonyl of said Thr, and R1 and R2 are independently H or an alkyl group of 1-12 carbons in length.

24. The personal care composition of claim 15, wherein the sugar amine is N-acetyl-D-glucosamine.

25. The personal care composition of claim 15 further comprising a sugar amine.

26. The personal care composition of claim 25 wherein said sugar amine is N-acetyl glucosamine.

27. The personal care composition of claim 16 wherein the hexamidine compound is hexamidine diisethionate.

28. A personal care composition comprising:
(a) a dipeptide having the following structure: 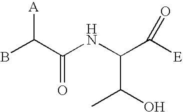
wherein
A=NH₃⁺(CH₂)₄—,
NH₂⁺═C(NH₂)NH—(CH₂)₃— or 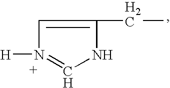
B=—NH₂, —NH₃⁺, —NH-D,
D=an acyl group of 2-22 carbon atoms in length, or biotinyl group and
E=—O-Alk, —NR₁R₂, —H, —O⁻, or —OH,
wherein Alk is an alkyl group of 1-24 carbons in length, and R₁ and R₂ are independently H or an alkyl group of 1-12 carbons in length; and
(b) a pentapeptide;
(c) a hexamindine compound;
(d) a vitamin B3 compound; and
(e) a dermatologically acceptable carrier.

29. The personal care composition of claim 28 wherein the vitamin B3 compound is niacinamide and derivatives thereof.

30. The personal care composition of claim 28 wherein the peptapeptide is palmitoyl-lys-thr-thr-lys-ser.

31. The personal care composition of claim 28 further comprising a sugar amine.

32. The personal care composition of claim 31 wherein said sugar amine is N-acetyl glucosamine.

33. The personal care composition of claim 28 wherein the hexamidine compound is hexamidine diisethionate.
